(19) United States
(12) Patent Application Publication
(10) Pub. No.: US 2015/0157753 A1
(43) Pub. Date: Jun. 11, 2015

(54) SYSTEM FOR DISINFECTION AND DEODORIZATION OF GARBAGE COLLECTION UNITS

(71) Applicant: Otri Comservice, Moscow (RU)

(72) Inventor: Yevgeniy Genadievich Sedov, Moscow (RU)

(21) Appl. No.: 14/532,867

(22) Filed: Nov. 4, 2014

(30) Foreign Application Priority Data
Dec. 9, 2013 (RU) RU2013154401

(51) Int. Cl. A61L 2/20

(52) U.S. Cl. CPC A61L 2/202

(57) ABSTRACT

A system for disinfecting and deodorizing garbage chutes using methane sensors at the top and/or bottom of the trash chute that includes an ozone generating unit at the bottom of the trash chute. The ozone-generating unit includes a flexible ozone distribution hose that emits ozone directly into the trash receptacle upon methane gases beginning to exceed the 50 parts per million threshold. Upon methane concentration levels reaching 50 parts per million, the ozone generator begins emitting ozone, the methane sensor continuously monitors ozone levels to ensure that there is no more residual methane left in the trash chute that exceeds a concentration of 50 parts per million. Optionally, the system can include an ozone sensor as a safety device to ensure that ozone levels do not surpass 0.09 parts per million.

### FIELD OF THE INVENTION

The present invention relates to the treatment of garbage chutes, and more particularly to treatment of waste in garbage chutes of multi-story buildings using effective and safe levels ozone to reduce or eliminate harmful methane levels.

### DESCRIPTION OF THE RELATED ART

Several designs for garbage disinfection systems using ozone have been designed in the past. None of them, however, include the use of methane sensors at the top and bottom of a trash chute to detect methane levels and then use corona discharge to deodorize and disinfect the trash chute. Additionally, none of them emit corona discharge ozone generation intermittently within the trash chute to reduce unnecessary exposure to ozone while still providing an effective amount. Also, none of the related art has found a way to focus the disinfection at the most intense part of the waste decomposition process to ensure efficient cleaning while minimizing dangrerous ozone exposure, all while also being able to disinfect and deodorize residual decomposition.

Applicant believes that the closest reference corresponds to PCT Application number PCT/LT2011/000001 (Publ. No. WO2011102701) issued to Cicelis, Rolandas. The Rolandas application discloses of a system to distribute ozone throughout a garbage collection system. The Rolandas reference further teaches of a remote means to operate the system and the installation of several ozone generators at unspecified locations along the garbage collection system. Moreover, the Rolandas reference discloses a way to introduce ozone into the garbage collection system at certain unspecified doses and at certain unspecified intervals depending on the size of the garbage collection, the ambient temperature and the frequency of emptying the rubbish container.

However, it differs from the present invention in many because the Rolandas reference fails to disclose or motivate several critical and beneficial features, which essentially affect the efficiency and effectiveness of garbage treatment in garbage chutes. First, by relying on the dimensions of the garbage chute, temperature and frequency of rubbish removal, the Rolandas leaves of the most important factor—the type of garbage found in the garbage collection unit. Garbage in garbage chutes is not standard; their organic and inorganic composition varies throughout the day, irrespective of temperature, dimensions or garbage removal frequency. Different types of garbage will affect the moisture and methane levels, thereby affecting the garbage chute conditions, rates of decomposition, and resulting foul odors.

Therefore, in the system disclosed in Rolandas both the overdose of ozone and the lack of it for the treatment are possible. The overdose is especially dangerous because large amounts of ozone are toxic for humans. The present invention uses methane detectors strategically places adjacent the garbage container to trigger the ozone generator at the onset of methane being released into the trash chute. Further, the system in the present invention has another methane detector strategically placed at the top of the garbage chute to detect any residual methane and again trigger the corona discharge ozone generator to precisely and safely disinfect and deodorize any remaining methane. This allows for efficient disinfecting and deodorizing of garbage collection units in multi-story building while eliminating the effect of the above-mentioned factors.

Other documents describing the closest subject matter provide for a number of more or less complicated features that fail to solve the problem in an efficient and economical way. None of these patents suggest the novel features of the present invention.

### SUMMARY OF THE INVENTION

It is one of the main objects of the present invention to provide a garbage chute treatment system that provides sufficient ozone for the destruction of bacteria and reduction of ammonium and hydrogen that cause foul odors, while minimizing human exposure to potentially hazardous ozone levels.

It is still another object of the present invention to provide a garbage chute treatment system that destroys insects and other infection-carrying organisms.

It is another object of this invention to provide at least two sensors strategically placed throughout a trash chute to efficiently reduce methane levels by emitting ozone at the first detection of methane levels and also being able to detect and address residual methane levels.

It is yet another object of this invention to provide such a system that is inexpensive to manufacture and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 represents a diagram showing the operating environment of the present invention with connections between bottom ozone generator 5, top methane detector 7, and control relay 8. The general path in which ozone can travel is represented using arrows.

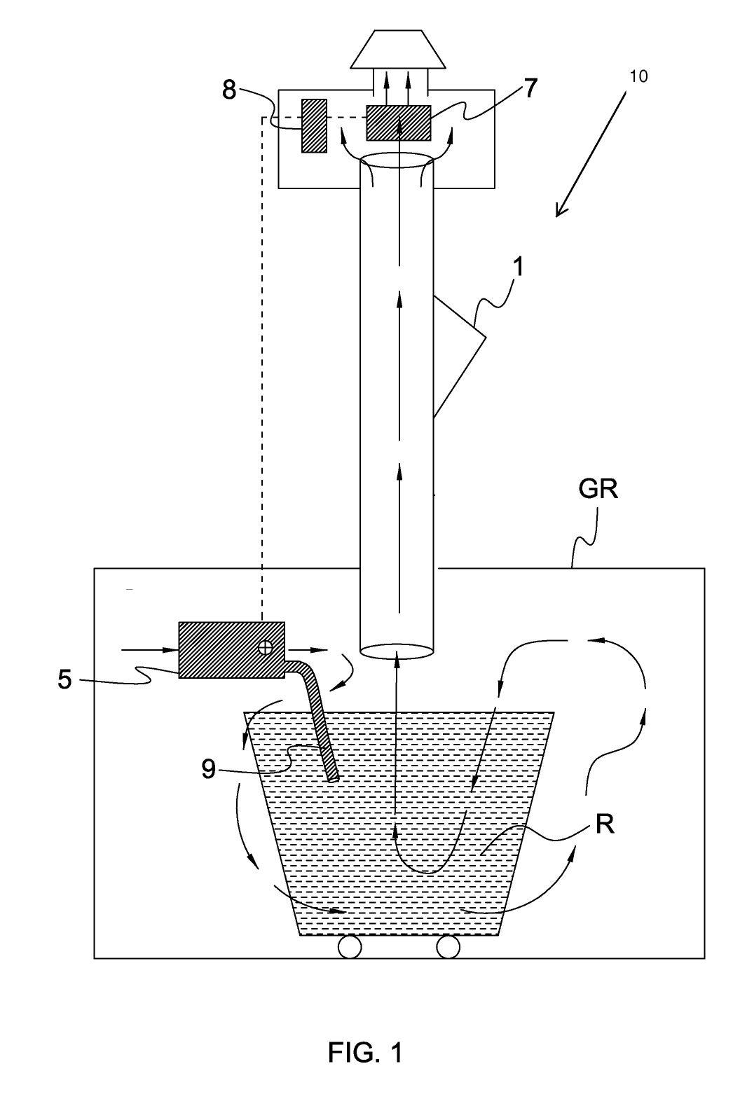

FIG. 2 shows an isometric view of ozone generating unit 5 having power indicator 9a.

FIG. 3 illustrates a front elevational view of ozone generating unit 5 showing filtered air-inlet 10. Distribution hose 9 is not shown as it is in the rear of ozone generating unit 5.

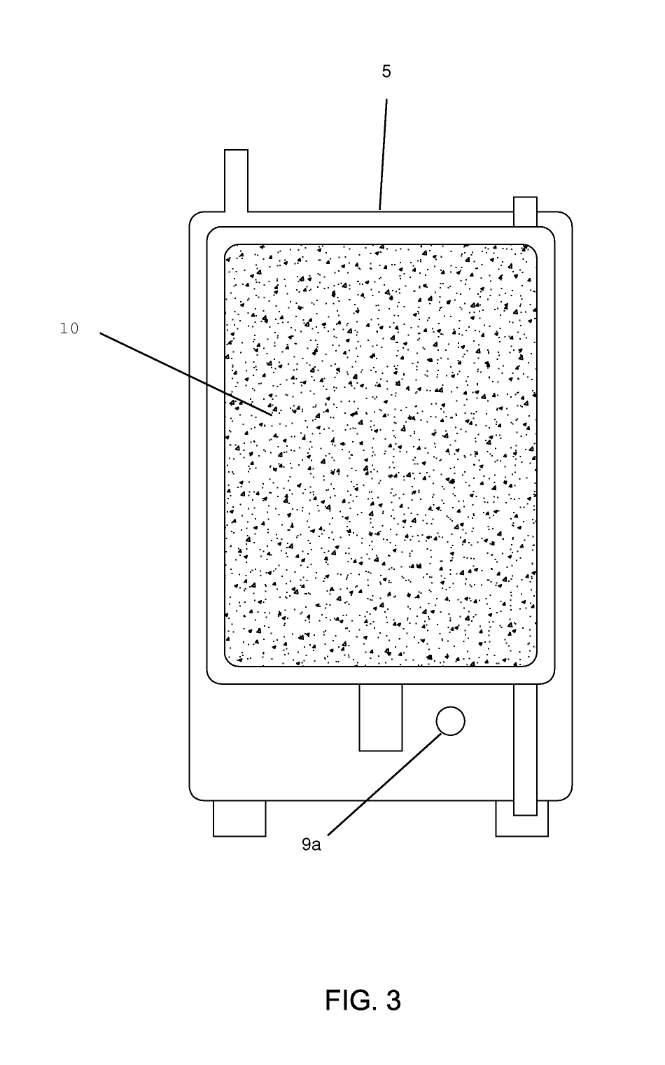

FIG. 4 is a diagram showing the operating environment of the present invention including ozone sensor 12 that can be placed in the stairwell of a building to measure the amount of ozone in the system.

### DETAILED DESCRIPTION OF THE EMBODIMENTS OF THE INVENTION

Referring now to the drawings, where the present invention is generally referred to with numeral 10, it can be observed that it basically includes top methane detecting unit 7 and control relay 8 located at the top of trash shaft 1. The system 10 further includes ozone generator 5 connected to ozone distribution hose 9 that emits ozone directly into trash receptacle R to kill bacteria and neutralize foul odors. Trash is thrown into shaft 1 from floors at different points throughout the length of shaft 1. Thrown out trash accumulates in receptacle R. Trash shaft 1 empties into receptacle R located in garbage room GR. Garbage room GR includes generating unit 5 located at the base of the trash shaft 1 and adjacent to trash receptacle R. Ozone generating unit 5 is precisely positioned adjacent receptacle R in order to directly feed ozone into the waste that produces elevating methane levels. Ozone generating unit 5 further includes ozone distributor hose 9 that is made of a flexible material and can be easily positioned outside of receptacle R when the trash is emptied.

When the quantity or type of trash in receptacle R causes methane levels to reach 50 parts per million (ppm), methane detector opening 6a allows methane to be measured by methane detector 6 to relay a signal to ozone generating unit 5 to begin emitting ozone into receptacle R. The ozone is released in a pulsating manner until methane levels in the garbage collection system are under 50 ppm. Top methane detecting unit 7 only relays a signal to ozone generating unit 5 through control relay 8 if methane levels in the top portion of trash shaft 1 reach 50 parts per million.

Top methane detector 7 continuously monitors methane levels in trash shaft 1. If residual methane levels above 50 parts per million are detected by top methane detector 7 bottom ozone generator 5 continues to emit ozone. Regardless of the temperature, dimensions, or frequency of garbage removal, ozone emission is immediately ceased upon methane levels dropping below 50 ppm, to ensure that humans will not come in contact with unhealthy amounts of ozone.

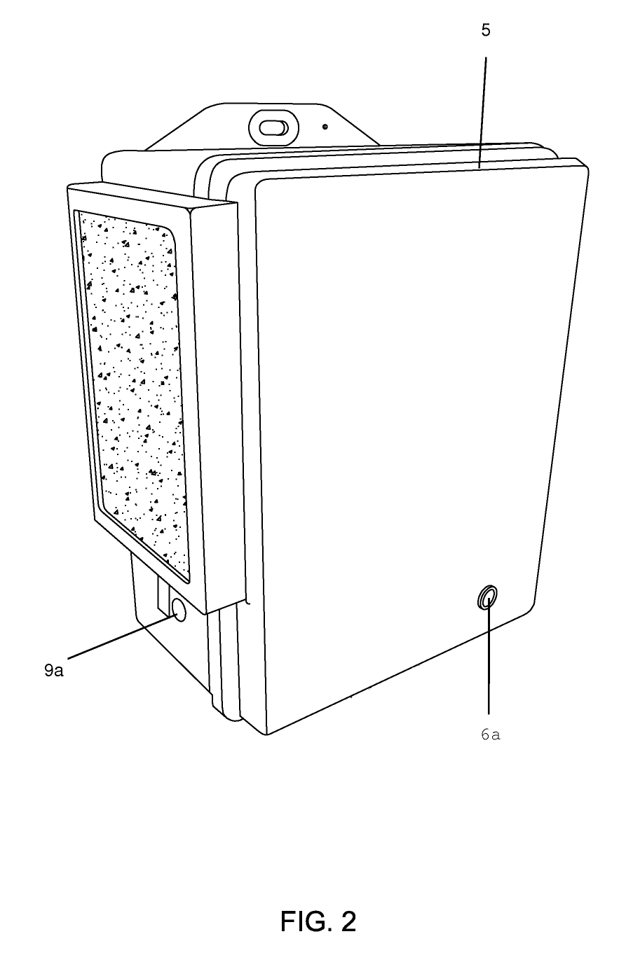

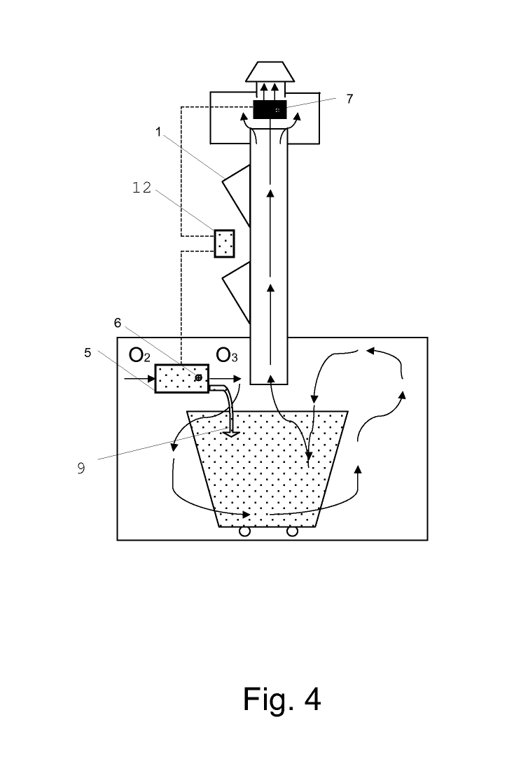

As shown in FIGS. 2, ozone generating unit 5 includes an methane detector opening 6a to allow methane to enter the methane sensors belonging to built-in methane detector 6. Ozone generating unit 5 further includes ozone unit power indicator 9a and flexible ozone distributor tube 9 that gets plugged into the rear of ozone generating unit 5. As shown in FIG. 4, the system in an alternate embodiment can include a safety feature consisting of ozone sensor 12 that continuously monitors the levels of ozone in the garbage chute 1 and garbage room GR so that ozone levels do not surpass 0.09 parts per million. Once, ozone levels drop to 0.01, ozone production will be resumed immediately. In one embodiment, control relay 8 is housed within top methane detector 7, further simplifying the system.

Time intervals between ozone emissions can be manually adjusted depending on the size of garbage room, the dimensions of the trash chute, and the outside temperature. However, the time intervals between ozone emissions can also be regulated using the ozone sensors and methane sensors. The ozone is generated and emitted into the trash chute until methane levels reach the desired level and as long as ozone levels are within a safe interval.

Ozone monitors can also be installed inside the stairwells of a building to measure the ozone levels throughout the day.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. A system for disinfecting and deodorizing garbage chutes, using at least one methane sensor assembly at an upper opening of said garbage chute, an ozone generator located adjacent a lower opening of said garbage chute, an ozone delivery tube mounted to said ozone generator emitting ozone directly into a trash receptable, and a control unit, thereby using said control unit to coordinate the emission of ozone through said ozone delivery tube when said methane sensor assembly records a methane concentration measurements of a predetermined parts per million quantity at said upper opening of said garbage chute.

2. The system in claim 1 wherein said ozone generators provide fifteen to twenty treatment cycles of ozone with one cycle every one to three minutes until methane concentration levels in the trash chute are below 50 parts per million.

3. The system in claim 1 wherein an ozone sensor is used to ensure that ozone production is discontinued upon ozone levels reaching 0.09 parts per million.

4. The system in claim 3 wherein ozone production is automatically reintroduced into said system upon ozone levels dropping to 0.01 parts per million.

5. The system set forth in claim 1 wherein said ozone delivery tube is made of a flexible material.

6. The system set forth in claim 1 wherein said control relay is built into said methane sensor assembly.

7. The system set forth in claim 1 wherein said predetermined parts per million quantity is 50 parts per million.

8. The system set forth in claim 1 wherein said ozone generator can be coordinated with said control relay and said methane detector using wired or wireless capabilities.

9. The system set forth in claim 1 wherein said ozone generator emits said treatment cycles in a pulsating manner.
